(19) United States
(12) Patent Application Publication
(10) Pub. No.: US 2025/0099031 A1
(43) Pub. Date: Mar. 27, 2025

(54) WEARABLE DEVICE DETECTING OBSTRUCTIVE SLEEP APNEA LEVEL INDEX AND INDEX DETERMINING METHOD THEREOF

(71) Applicant: PIXART IMAGING INC., Hsin-Chu County (TW)

(72) Inventors: Chih-Hao WANG, Hsin-Chu County (TW); Shih-Jen LU, Hsin-Chu County (TW); Chien-Yi KAO, Hsin-Chu County (TW); Yang-Ming CHOU, Hsin-Chu County (TW); Hsin-Yi LIN, Hsin-Chu County (TW)

(21) Appl. No.: 18/413,320

(22) Filed: Jan. 16, 2024

Related U.S. Application Data
(60) Provisional application No. 63/539,863, filed on Sep. 22, 2023.

(51) Int. Cl. A61B 5/00; A61B 5/1455

(52) U.S. Cl. CPC A61B 5/4818; A61B 5/14552; A61B 5/681; A61B 5/725; A61B 5/7278; A61B 5/7282; A61B 5/743; A61B 2562/0242

(57) ABSTRACT

There is provided a wearable device including at least one light source, a light sensor and a processor. The processor generates a peak interval plot according to one of at least two light detection signals detected by the light sensor when the at least one light source emits light, and generates an oxygen saturation plot according to two of the one of at least two light detection signals detected by the light sensor when the at least one emits light. The processor further determines an Apnea Hypopnea Index (AHI) score according to the peak interval plot, determines an Oxygen Desaturation Index (ODI) score according to the oxygen saturation plot and fits an obstructive sleep apnea level index corresponding to the AHI score and the ODI score.

### CROSS-REFERENCE TO RELATED APPLICATIONS

The present application claims the priority benefit of U.S. Provisional Application Ser. Number U.S. 63/539,863, filed on Sep. 22, 2023, the disclosure of which is hereby incorporated by reference herein in its entirety.

### FIELD OF THE DISCLOSURE

This disclosure generally relates to an optical detecting device and, more particularly, to a wearable device adapted to detecting an obstructive sleep apnea level index optically and index determining methods thereof.

### BACKGROUND OF THE DISCLOSURE

Current standard methods for evaluating the obstructive sleep apnea (OSA) is to conduct a polysomnography (PSG) test, which measures the breathing air flow, electroencephalogram (EEG), SpO₂, respiration movement and electrocardiogram the whole sleep period, and these test data are analyzed later by experts to determine an OSA level index. This standard procedure is generally executed in institutions such as hospitals or sleeping centers. Although users may operate a simplified PSG device by him/herself at home to evaluate the OSA risk, a nasal flow meter still needs to be equipped in sleeping. However, the nasal flow meter can make the users feel uncomfortable to degrade the sleeping quality.

### SUMMARY

Accordingly, the present disclosure provides a wearable device adapted to detecting an obstructive sleep apnea (OSA) level index and index determining methods thereof. A user does not need to equip any nasal flow meter during measurement such that the sleeping quality is not influenced.

The present disclosure provides a wearable device and index determining methods thereof that use light sources of at least two light wavelengths to obtain a peak interval plot and a blood oxygen saturation (i.e. SpO₂) plot that are respectively used to determine an Apnea Hypopnea Index (AHI) score and an Oxygen Desaturation Index (ODI) score. The OSA level index is obtained from the AHI score and the ODI score by using a predetermined machine learning model and parameters. By using the wearable device of the present disclosure, a user may perform the long-term recording easily.

The present disclosure provides a wearable device for acquiring an obstructive sleep apnea (OSA) level index of a user. The wearable device includes a device main body, at least one light source, a light sensor and a processor. The device main body has an inner surface configured to be attached to a surface of a skin of the user. The at least one light source is arranged on the inner surface and configured to emit light of at least two wavelengths. The light sensor is arranged on the inner surface and configured to output at least two photoplethysmography (PPG) signals in response to light emission of the at least one light source. The processor is configured to generate a peak interval plot according to one of the at least two PPG signals, generate an oxygen saturation (SpO2) plot according to two of the at least two PPG signals, determine an Apnea Hypopnea Index (AHI) score according to the peak interval plot, determine an Oxygen Desaturation Index (ODI) score according to the SpO2 plot, and determine the OSA level index according to the AHI score and the ODI score.

The present disclosure further provides a determining method of an AHI score, including the steps of: labelling reasonable peak intervals to determine a peak interval plot; determining a steady segment in the peak interval plot; up-sampling peak interval data within the steady segment to generate up-sampled interval data; respectively calculating a difference between every adjacent data points of the up-sampled interval data to generate interval difference data; calculating variances of the interval difference data to generate variance data; determining a first threshold according to the variance data and the peak interval data; determining a second threshold according to the first threshold and a number of consecutive data points in the variance data smaller than the first threshold; and taking a ratio of a counting number of the variance data larger than the second threshold and a total time of the peak interval plot as the AHI score.

The present disclosure provides a determining method of an ODI score, including the steps of: scanning the SpO2 plot to determine a reasonable SpO2 value; determining a base SpO2 according to SpO2 values within a predetermined time behind the reasonable SpO2 value; determining a low SpO2 threshold according to the base SpO2; and taking a ratio of a counting value of consecutive data points behind the reasonable SpO2 value in the SpO2 plot lower than the low SpO2 threshold and a total time of the SpO2 plot as the ODI score.

### BRIEF DESCRIPTION OF DRAWINGS

Other objects, advantages, and novel features of the present disclosure will become more apparent from the following detailed description when taken in conjunction with the accompanying drawings.

FIG. 1A is a schematic diagram of a wearable device adapted to detecting an OSA level index according to one embodiment of the present disclosure.

FIG. 1B is an operational schematic diagram of a wearable device adapted to detecting an OSA level index according to one embodiment of the present disclosure.

FIG. 2 is a schematic block diagram of a wearable device adapted to detecting an OSA level index according to one embodiment of the present disclosure.

FIG. 3 is a schematic diagram of a peak interval plot obtained by a wearable device adapted to detecting an OSA level index according to one embodiment of the present disclosure.

FIG. 4 is a schematic diagram of an SpO₂ plot obtained by a wearable device adapted to detecting an OSA level index according to one embodiment of the present disclosure.

FIG. 5 is a flow chat of an AHI score obtained by a wearable device adapted to detecting an OSA level index according to one embodiment of the present disclosure.

FIG. 6A is a schematic diagram of data points of the Steps S513 to S515 in FIG. 5.

FIG. 6B is a schematic diagram of data points of the Step S517 in FIG. 5.

FIG. 7 is a flow chat of an ODI score obtained by a wearable device adapted to detecting an OSA level index according to one embodiment of the present disclosure.

FIG. 8 is a schematic diagram of determining an OSA level index according to an AHI score and an ODI score obtained by a wearable device adapted to detecting an OSA level index according to one embodiment of the present disclosure.

FIG. 9 is a schematic diagram of a conversion model of an OSA level index from the AHI score and the ODI score in FIG. 8.

### DETAILED DESCRIPTION OF THE DISCLOSURE

It should be noted that, wherever possible, the same reference numbers will be used throughout the drawings to refer to the same or like parts.

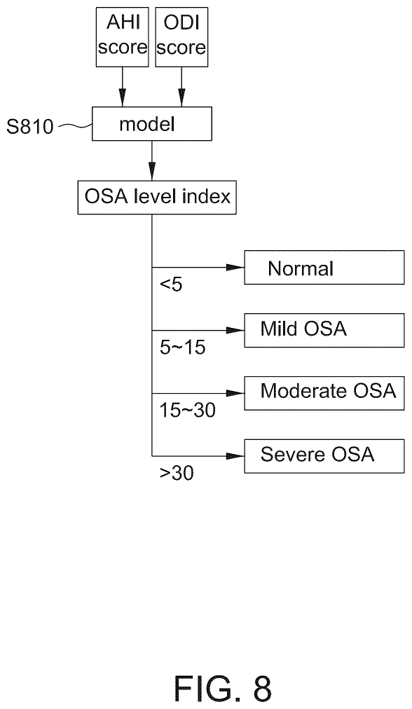

One objective of the present disclosure is to provide a wearable device capable of obtaining an obstructive sleep apnea (OSA) level index using a full-optical method, i.e. without using a nasal flow meter. The wearable device includes a light sensor for detecting photoplethysmography (PPG) signals associated with different light wavelengths. The PPG signals are used to generate an Apnea Hypopnea Index (AHI) score and an Oxygen Desaturation Index (ODI) score, which are then fitted to an OSA level index using a predetermined model and parameters. The method of determining an OSA category according to an OSA level index is known to the art, e.g., referring to FIG. 8, and thus details thereof are not described herein.

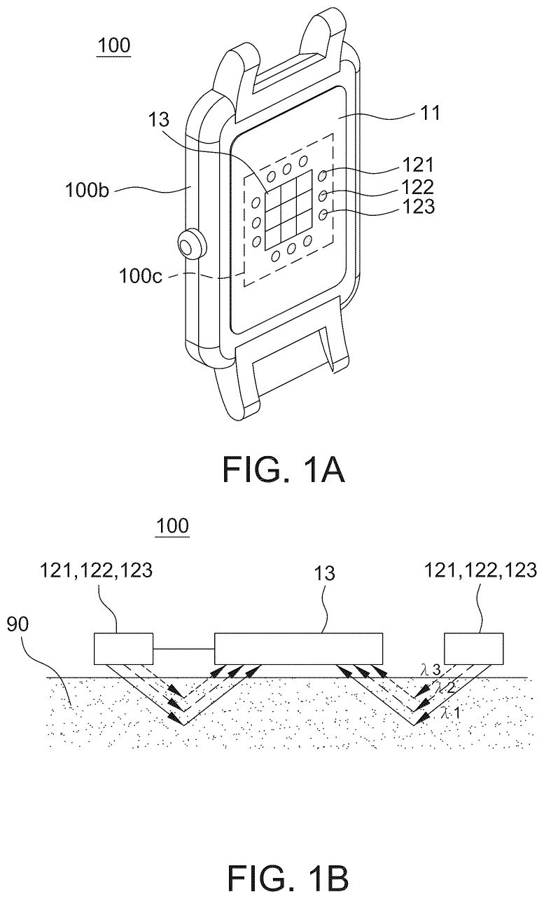

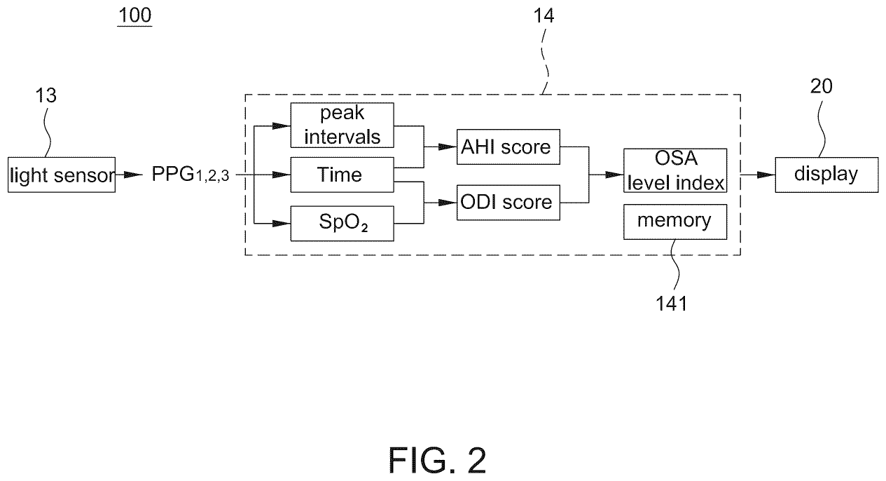

Please refer to FIGS. 1A, 1B and 2, FIG. 1A is a schematic diagram of a wearable device 100 adapted to detecting an OSA level index according to one embodiment of the present disclosure; FIG. 1B is an operational schematic diagram of a wearable device 100 adapted to detecting an OSA level index according to one embodiment of the present disclosure; and FIG. 2 is a schematic block diagram of a wearable device 100 adapted to detecting an OSA level index according to one embodiment of the present disclosure.

The wearable device 100 is selected from an electronic device or accessory that is a contact with a skin 90 when it is worn on a user's body, such as a watch, a bracelet, a ring, an ear plug, and a sleep mask, but not limited to. The wearable device 100 includes a device main body 100b and a sensor chip 100c as shown in FIG. 1A.

The device main body 100b has an inner surface 11 (referring to FIG. 1A) which is used to be attached to a surface of the skin 90, wherein a location of the skin 90 in a human body is determined according to a type of the wearable device 100.

The sensor chip 100c includes a first light source 121, a second light source 122, a third light source 123, a light sensor 13 and a processor 14, but the present disclosure is not limited to. In another aspect, at least one of the first light source 121, the second light source 122, the third light source 123, the light sensor 13 and the processor 14 is arranged in a different chip or module.

The first light source 121, the second light source 122 and the third light source 123 are, for example, light emitting diodes or laser diodes, and are arranged on the inner surface 11 respectively for emitting light of a first wavelength λ1, light of a second wavelength λ2 and light of a third wavelength λ3 toward the skin 90, wherein the first wavelength λ1, the second wavelength λ2 and the third wavelength λ3 are different from one another. For example, the light of the first wavelength λ1 is green light, the light of the second wavelength λ2 is red light and the light of the third wavelength λ3 is infrared light, but not limited thereto.

The light sensor 13 is, for example, a complementary metal oxide semiconductor (CMOS) image sensor or a single photon avalanche photodiode (SPAD) image sensor, but not limited to. The light sensor 13 is arranged on the inner surface 11 and facing the skin 90. The light sensor 13 is used to output a first PPG signal PPG₁ corresponding to light emission of the first light source 121, output a second PPG signal PPG₂ corresponding to light emission of the second light source 122, and output a third PPG signal PPG₃ corresponding to light emission of the third light source 123. In one aspect, the first light source 121, the second light source 122 and the third light source 123 emit light sequentially. In another aspect, in the case that the light sensor 13 is able to distinguish light energy from different light sources (e.g., arranged with filter(s), or different light sources emitting light having different features), the first light source 121, the second light source 122 and the third light source 123 emits light simultaneously.

It should be mentioned that although FIG. 1A shows that the first light source 121, the second light source 122 and the third light source 123 are arranged at four sides of the light sensor 13, the present disclosure is not limited thereto. The light sensor 13 outputs the PPG₁, PPG₂ and PPG₃ as long as the first light source 121, the second light source 122 and the third light source 123 are arranged at positions that can emit light toward to the skin 90 and back to the light sensor 13. In one aspect, at least one of the first light source 121, the second light source 122 and the third light source 123 is arranged adjacent to different sides of the light sensor 13. In another aspect, at least one of the first light source 121, the second light source 122 and the third light source 123 are arranged to have a different distance from the light sensor 13.

The processor 14 is, for example, a digital signal processor (DSP), an application specific integrated circuit (ASIC), a micro controller unit (MCU) or a field programmable gate array (FPGA), and the processor 14 implements functions thereof, e.g., including calculating an AHI score, an ODI score and an OSA level index shown in FIG. 2, using software, firmware and/or hardware. The processor 14 is coupled to the light sensor 13 to receive the first PPG signal PPG₁, the second PPG signal PPG₂ and the third PPG signal PPG₃, and determines the OSA level index according to these PPG signals PPG₁, PPG₂ and PPG₃, e.g., described in FIG.4 and FIGS. 7-8 by an example.

The wearable device 100 further includes a display 20 or is wirelessly coupled to an electronic device having the display 20. The display 20 is used to show the OSA level index (e.g., showing values of the OSA level index) or to show an OSA category associated with the OSA level index, e.g., FIG. 8 showing categories including a normal, a mild OSA, a moderate OSA and a severe OSA, such that a user may aware of the evaluated OSA risk through the display 20. The OSA level index and the OSA category may be recorded in a memory 141 for being accessed by an external device or an external system via wired or wireless communication.

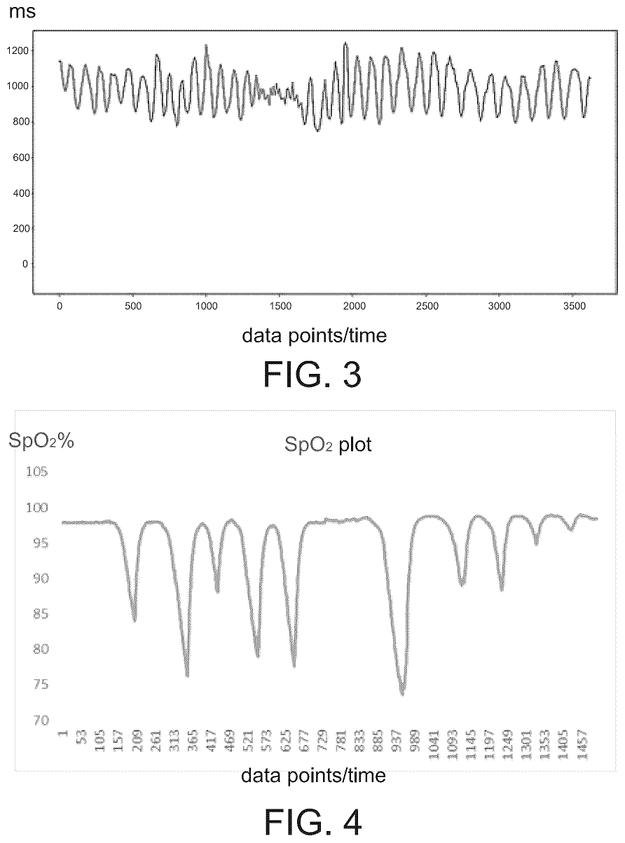

For example, the processor 14 generates a peak interval plot (e.g., referring to FIG. 3) according to the first PPG signal PPG₁, generates an oxygen saturation plot according to the second PPG signal PPG₂ and the third PPG signal PPG₃ (e.g., referring to SpO₂ plot shown in FIG. 4), determines an Apnea Hypopnea Index (AHI) score according to the peak interval plot, determines an Oxygen Desaturation Index (ODI) score according to the SpO₂ plot, and determines an OSA level index according to the AHI score and the ODI score. In the present disclosure, the processor 14 is to calculate a number of AHI events and ODI events occurred within a total recording time (sometimes abbreviated as total time herein), the processor 14 further receives data point time (shown as Time in FIG. 2) in determining the AHI score and the ODI score.

In one aspect, the peak interval plot is formed by R-R intervals (milliseconds) in heart rate waveforms at different times, wherein the meaning of an R-R interval in a heart rate waveform is known to the art and thus details thereof are not described herein. Furthermore, the method of calculating the SpO₂ using PPG signals associated with two light wavelengths is known to the art, and thus details thereof are not described herein. In the present disclosure, the light wavelengths λ1, λ2 and λ3 are respectively referred to a dominant wavelength of a light spectrum. That is, the light sensor 13 provides at least two PPG signals associated with at least two light wavelength ranges for the processor 14 to respectively calculate peak intervals and SpO₂. In calculating the SpO₂, the two light wavelength ranges being used are partially overlapped or not overlapped. It is possible to use a same light wavelength range to respectively calculate the peak interval plot and the SpO₂ plot.

In the present disclosure, the AHI score indicates a number of times that sleep apnea occurs within a total recording time (e.g., determined by a user manually controlling the start of recording before sleep and manually controlling the stop of the recording after wakeup, or determined by the wearable device 100 that has a function of automatically identifying or pre-setting the start and stop of recording) of the peak interval plot. For example, the processor 14 counts a number of times that a variance of peak interval differences within the total recording time exceeds a threshold region (described by an example below) as the number of times that the sleep apnea occurs, wherein the threshold region is determined according to each peak interval plot generated each time, e.g., the peak interval plot and the threshold region measured every night being different from one another.

In the present disclosure, the ODI score is a number of times that oxygen desaturation occurs within a total recording time of the SpO₂ plot. For example, the processor 14 counts a number of times that consecutive data points of SpO₂ within the total recording time are smaller than a low SpO₂ threshold as the number of times that the oxygen desaturation occurs, wherein the low SpO₂ threshold is determined according to each SpO₂ plot generated each time, e.g., the SpO₂ plot and the low SpO₂ threshold measured every night being different from one another.

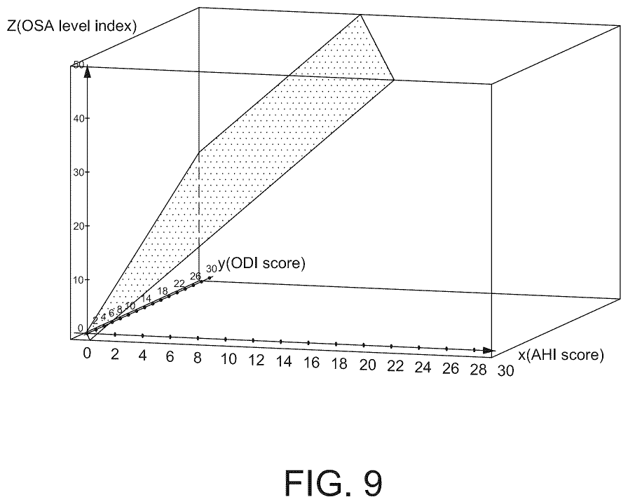

Finally, the processor 14 converts/fits the AHI score and the ODI score to an OSA level index using a predetermined machine learning model, e.g., shown as S810 in FIG. 8. The predetermined machine learning model is generated based on the partial least square (PLS), principal components (PC), multiple linear regression (MLR), artificial neural network (ANN), K-nearest neighbor (KNN), decision tree (DT), support vector machine (SVM), but not limited to. The parameters of the predetermined machine learning model can also be generated based on the partial least square (PLS), principal components (PC), multiple linear regression (MLR), artificial neural network (ANN), K-nearest neighbor (KNN), decision tree (DT), support vector machine (SVM), and be updated by edge training later. For example, FIG. 9 shows a method for converting/fitting the AHI score and the ODI score to an OSA level index using the MLR. It is seen from FIG. 9 that when one pair of AHI score and ODI score is determined, one OSA level index is determined accordingly.

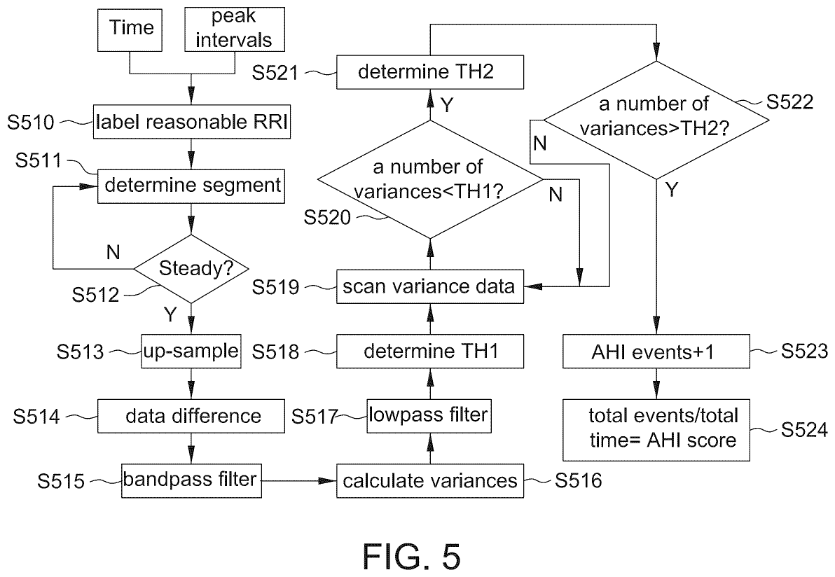

Please refer to FIGS. 2 and 5, an example of a determining method of an AHI score is described hereinafter.

The processor 14 calculates the AHI score according to the peak interval plot and corresponding recording times.

Step S510: The processor 14 labels reasonable peak intervals in PPG₁ to determine a peak interval plot. In one aspect, when a peak interval is an R-R interval of a heart rate waveform, the reasonable peak intervals include peak intervals between 400 ms and 1800 ms.

Step S511-S512: Next, the processor 14 determines a steady segment in the peak interval plot. In processing the whole peak interval plot, multiple steady segments may be determined. In one aspect, the steady segment is arranged as a difference between adjacent data points of the peak interval data being smaller than 2 times of a standard deviation of the peak interval data in the reasonable peak interval. In brief, the steady segment is referred to a variation of peak interval data thereof being within a predetermined range. The steady segment is to avoid the motion interference (e.g., turning the body over) in sleeping. When the difference between adjacent data points of the peak interval data is larger than a predetermined range (e.g., 2 times of the standard deviation), the processor 14 generates one steady segment for being processed by the following steps. That is, the Steps 513-S523 below is performed sequentially on each steady segment.

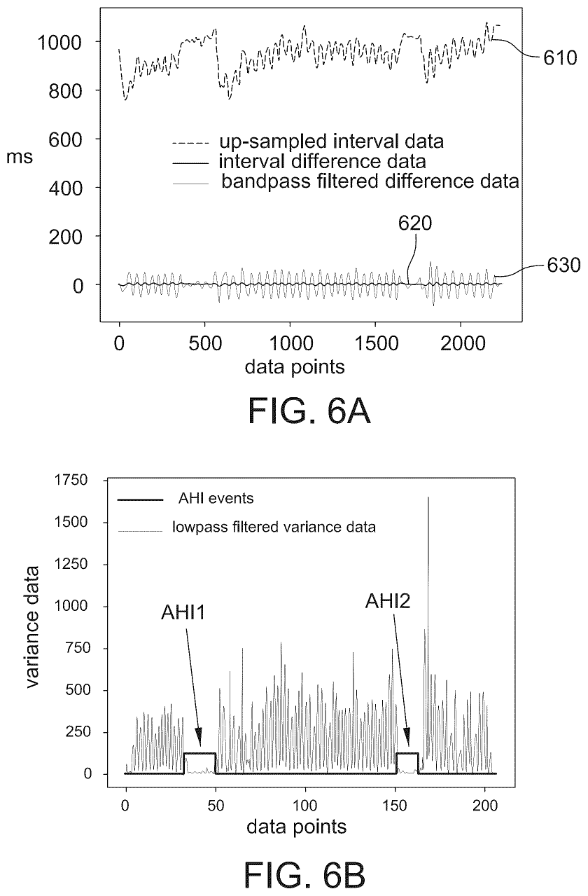

Step S513: To increase the calculation accuracy, the processor 14 performs up-sampling on the peak interval data in the steady segment to generate up-sampled interval data, such that a capturing frequency of PPG signals of the light sensor 13 may be reduced. In one aspect, the up-sampling rate is selected from 10 to 40 times of the peak interval data. For example, FIG. 6A shows the up-sampled interval data 610.

Step S514: The processor 14 then respectively calculates a difference between every adjacent data points of the up-sampled interval data to generate interval difference data, e.g., FIG. 6A showing the interval difference data 620. The present disclosure is to use the difference of the up-sampled interval data (i.e. variation with time) to identify whether sleep apnea occurs.

Step S516: The processor 14 calculates variances of the interval difference data to generate variance data, e.g., calculating square of standard deviation of the interval difference data, but not limited to. To remove noises and improve accuracy, the processor 14 firstly performs the bandpass filtering on the interval difference 620 (Step S515) and then calculates the variance data. FIG. 6A shows the bandpass filtered difference data 630.

Step S517: The processor 14 determines a first threshold TH1 according to the variance data (or the bandpass filtered difference data 630) and the peak interval data. In one aspect, the first threshold TH1 is determined according to an average Avg1 of the variance data (or the bandpass filtered difference data 630) and an average Avg2 of the peak interval data of the steady segment, e.g., (Avg1/Avg2)×N, wherein N is a predetermined positive value that can be derived from clinical data of PPG with apneas and hypopneas (for example, N is between 500-1000). To remove noises and improve accuracy, the processor 14 firstly performs the lowpass filtering on the variance data (or the bandpass filtered difference data 630) (Step S517) and then calculates the first threshold TH1, e.g., FIG. 6B showing the lowpass filtered variance data.

Step S519 to S521: The processor 14 determines a second threshold TH2 according to the first threshold TH1 and a number of consecutive data points in the variance data smaller than the first threshold TH1. For example, if a number of consecutive data points that are smaller than the first threshold TH1 is M, TH2=TH1×M. That is, the second threshold TH2 is positively correlated with the number of consecutive data points smaller than the first threshold TH1. A value of the second threshold TH2 is higher when the value of M is higher.

Step S522 to S524: The processor 14 takes a ratio of a counting number of the variance data (or lowpass filtered variance data) larger than the second threshold TH2 and a total time of the peak interval plot as the AHI score. For example, FIG. 6B shows that two AHI events AHI1 and AHI2 appear in one steady segment, and thus the processor 14 adds 2 to the apnea event in the steady segment. It should be mentioned that a value of AHI event in Step S523 is increased by 1 when one sleep apnea appears. That is, the AHI score is a total number of AHI events divided by a total time of the peak interval plot.

The processor 14 updates an accumulated value of the AHI event obtained in Step S523 corresponding to each steady segment (if OSA event appearing). A length of the total time is determined by a total sleeping time of one night (assuming the measurement performed at night). The wearable device 100 of the present disclosure may start to record when a user starts sleeping, and the processor 14 takes the whole recording time as the total time mentioned herein, or removes a predetermined time interval (e.g., 5 to 10 minutes) in the beginning (just fall asleep) and the ending (before getting up) from the whole recording time as the total time mentioned herein.

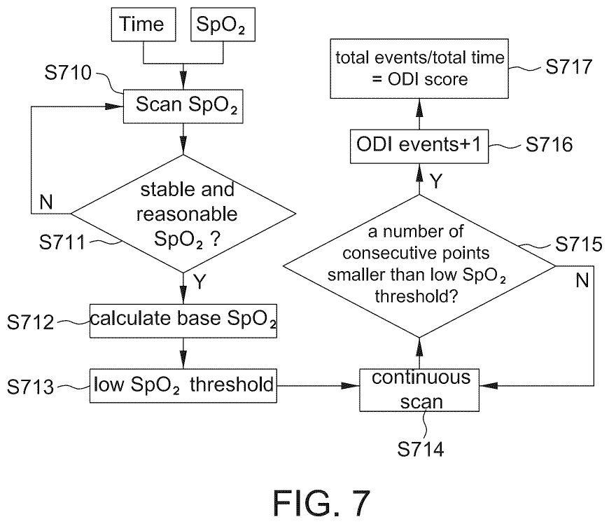

Please refer to FIGS. 2 and 7, an example of a determining method of an ODI score is described hereinafter.

The processor 14 calculates the ODI score according to the SpO₂ plot and corresponding recording times.

Step S710-S711: The processor 14 scans the SpO2 plot to determine a reasonable SpO₂ value. In one aspect, the reasonable SpO₂ value is arranged as a standard deviation within a predetermined time (e.g., one to two minutes) of the SpO₂ plot being smaller than 2, but not limited to 2. The next step is entered only after the processor 14 confirms the appearance of the reasonable SpO₂ value. In one aspect, the processor 14 executes the Step S711 only once within one SpO₂ plot (e.g., corresponding to one sleep).

Step S712: After the reasonable SpO₂ value appears, the processor 14 then determines a base SpO₂ according to SpO₂ values (considered as reasonable values) within a predetermined time (e.g., one to two minutes). In one aspect, the base SpO₂ is an average of the SpO₂ values within the predetermined time, but not limited to. In the present disclosure, the base SpO₂ may be different corresponding to each recorded SpO₂ plot (e.g., corresponding to each sleep).

Step S713: After the base SpO₂ is determined, the processor determines a low SpO₂ threshold according to the base SpO₂. In one aspect, the low SpO₂ threshold is arranged as 2% to 5% of the base SpO₂, but not limited to.

Steps S714 to S716: In the present disclosure, in scanning the SpO₂ values after the appearance of the reasonable SpO₂ value, when there are a predetermined number of consecutive data points in SpO₂ values of the SpO₂ plot being smaller than the low SpO₂ threshold, the processor 14 adds 1 to a number of ODI events (Step S716). In one aspect, the predetermined number of the consecutive data points is larger than or equal to 4 data points, but not limited to 4. In other words, when a number of consecutive data points smaller than the low SpO₂ threshold is smaller than 4, a value of the ODI event is not increased by 1.

Step S717: Finally, the processor 14 takes a ratio of a counting value of consecutive data points of SpO₂ value (considered reasonable values herein) in the SpO₂ plot lower than the low SpO₂ threshold and a total time of the SpO₂ plot as the ODI score. That is, the ODI score is a number of total ODI events divided by a total time of the SpO₂ plot.

Although FIG. 7 does not show up-sampling the SpO₂ data, the present disclosure is not limited thereto. The processor 14 may perform the up-sampling before the Step S714.

In one aspect, the determining method of the AHI score and the ODI score may be processed by the processor 14 at the same time after the user gets up, e.g., all PPG signals (e.g., including PPG₁, PPG₂ and PPG₃) measured by the light sensor 13 in a sleeping are all recorded in the memory 141. In another aspect, the determining method of the AHI score and the ODI score may be processed by the processor 14 once every predetermined time interval (e.g., one to two hours), and the updated counting value (e.g., in the Steps S523 and S716) are stored in the memory 141, i.e. recording a number of AHI events and ODI events. The processor 14 calculates (after the sleeping) a ratio of a final AHI event value and a final ODI event value and the total time to obtain the AHI score and the ODI score, respectively.

It should be mentioned that although the above embodiments are described by an example having three light sources, the present disclosure is not limited thereto. In another aspect, the peak interval plot is obtained by using one of two light sources (e.g., red light) for generating the SpO₂ plot. In a further aspect, light of at least two wavelengths are generated by one light source, e.g., by adjusting the driving parameter(s) of the one light source to change the emission light wavelength.

It should be mentioned that the values, such as a number of data points, the time, the ratio, the percentage mentioned in the above embodiment is only intended to illustrate but not to limit the present disclosure.

As mentioned above, the conventional benchmark device for determining the OSA level index includes a nasal flow meter for being equipped by a user in sleeping such that the sleeping quality can be influenced. Accordingly, the present disclosure further provides a wearable device adapted to detecting the OSA level index full-optically (e.g., FIG. 2) and index determining methods thereof (e.g., FIG. 5 and FIGS. 7-8) that determine the AHI score and the ODI score using PPG signals associated with different light wavelengths, and accordingly determine a corresponding OSA level index using a predetermined model and parameters. The diagnostic result of detecting the OSA level index by the present disclosure is validated to have certain accuracy in comparison with test results of the conventional benchmark device with two general diagnostic thresholds, RI>5 and RI>15.

Although the disclosure has been explained in relation to its preferred embodiment, it is not used to limit the disclosure. It is to be understood that many other possible modifications and variations can be made by those skilled in the art without departing from the spirit and scope of the disclosure as hereinafter claimed.

1. A wearable device, configured to acquire an obstructive sleep apnea (OSA) level index of a user, the wearable device comprising:
a device main body, comprising an inner surface configured to be attached to a surface of a skin of the user;
at least one light source, arranged on the inner surface and configured to emit light of at least two wavelengths;
a light sensor, arranged on the inner surface and configured to output at least two photoplethysmography (PPG) signals in response to light emission of the at least one light source; and
a processor, configured to
generate a peak interval plot according to one of the at least two PPG signals,
generate an oxygen saturation (SpO₂) plot according to two of the at least two PPG signals,
determine an Apnea Hypopnea Index (AHI) score according to the peak interval plot,
determine an Oxygen Desaturation Index (ODI) score according to the SpO₂ plot, and
determine the OSA level index according to the AHI score and the ODI score.

2. The wearable device as claimed in claim 1, wherein
one of the light of the at least two wavelengths is red light, and
the other one of the light of the at least two wavelengths is infrared light.

3. The wearable device as claimed in claim 1, wherein
the wearable device further comprises a display or is wirelessly coupled to an electronic device having a display, and
the display is configured to show the OSA level index or an OSA category associated with the OSA level index.

4. The wearable device as claimed in claim 1, wherein the AHI score indicates a number of times that sleep apnea occurs within a total recording time of the peak interval plot.

5. The wearable device as claimed in claim 4, wherein the processor is further configured to
count a number of times that a variance of peak interval differences within the total recording time exceeds a threshold region as the number of times that the sleep apnea occurs.

6. The wearable device as claimed in claim 5, wherein the threshold region is determined according to each peak interval plot generated each time.

7. The wearable device as claimed in claim 1, wherein the ODI score is a number of times that oxygen desaturation occurs within a total recording time of the SpO₂ plot.

8. The wearable device as claimed in claim 7, wherein the processor is further configured to
count a number of times that consecutive data points of SpO₂ within the total recording time are smaller than a low SpO₂ threshold as the number of times that the oxygen desaturation occurs.

9. The wearable device as claimed in claim 8, wherein the low SpO₂ threshold is determined according to each SpO₂ plot generated each time.

10. The wearable device as claimed in claim 1, wherein the processor is further configured to
convert the AHI score and the ODI score to the OSA level index using a predetermined machine learning model.

11. A determining method of the AHI score of claim 1, the determining method comprising:
labelling reasonable peak intervals to determine the peak interval plot;
determining a steady segment in the peak interval plot;
up-sampling peak interval data within the steady segment to generate up-sampled interval data;
respectively calculating a difference between every adjacent data points of the up-sampled interval data to generate interval difference data;
calculating variances of the interval difference data to generate variance data;
determining a first threshold according to the variance data and the peak interval data;
determining a second threshold according to the first threshold and a number of consecutive data points in the variance data smaller than the first threshold; and
taking a ratio of a counting number of the variance data larger than the second threshold and a total time of the peak interval plot as the AHI score.

12. The determining method as claimed in claim 11, wherein the reasonable peak intervals comprising peak intervals between 400 ms and 1800 ms.

13. The determining method as claimed in claim 11, wherein the steady segment is arranged as a difference between adjacent data points of the peak interval data being smaller than 2 times of a standard deviation of the peak interval data in the reasonable peak interval.

14. The determining method as claimed in claim 11, wherein the first threshold is determined according to an average of the variance data and an average of the peak interval data of the steady segment.

15. The determining method as claimed in claim 11, wherein the second threshold is positively correlated with the number of consecutive data points smaller than the first threshold.

16. The determining method as claimed in claim 11, further comprising:
performing bandpass filtering on the interval difference data; and
performing low pass filtering on the variance data.

17. A determining method of the ODI score of claim 1, the determining method comprising:
scanning the SpO₂ plot to determine a reasonable SpO₂ value;
determining a base SpO₂ according to SpO₂ values within a predetermined time behind the reasonable SpO₂ value;
determining a low SpO₂ threshold according to the base SpO₂; and
taking a ratio of a counting value of consecutive data points behind the reasonable SpO₂ value in the SpO₂ plot lower than the low SpO₂ threshold and a total time of the SpO₂ plot as the ODI score.

18. The determining method as claimed in claim 17, wherein the reasonable SpO₂ value is arranged as a standard deviation within another predetermined time of the SpO₂ plot being smaller than 2.

19. The determining method as claimed in claim 17, wherein
the base SpO₂ is an average of the SpO₂ values within the predetermined time, and
the low SpO₂ threshold is arranged as 2% to 5% of the base SpO_{2.}

20. The determining method as claimed in claim 17, wherein a number of the consecutive data points is larger than or equal to 4 data points.
